(19) United States
(12) Patent Application Publication
(10) Pub. No.: US 2023/0240998 A1
(43) Pub. Date: Aug. 3, 2023

(54) TOFACITINIB EXTENDED RELEASE FORMULATIONS

(71) Applicant: Hikma Pharmaceuticals USA Inc., Berkeley Heights, NJ (US)

(72) Inventors: Nirav J. Patel, Plain City, OH (US); Sudhir Gorukanti, Dublin, OH (US); Andrea Cady, Dublin, OH (US); Phanidhara Kotamraj, Powell, OH (US); Todd Branch, Columbus, OH (US); Surya Prakash Rao Pydimarry, Plain City, OH (US); Praneeth Rao Kakullamarri, Powell, OH (US)

(21) Appl. No.: 18/068,631

(22) Filed: Dec. 20, 2022

Related U.S. Application Data
(60) Provisional application No. 63/400,108, filed on Aug. 23, 2022.
(60) Provisional application No. 63/265,884, filed on Dec. 22, 2021.

(51) Int. Cl. A61K 9/50; A61K 31/519; A61K 9/48

(52) U.S. Cl. CPC A61K 9/5026; A61K 9/5031; A61K 9/5047; A61K 31/519; A61K 9/4866

(57) ABSTRACT

Sustained release pharmaceutical dosage forms include a core containing at least one swellable, water soluble, pH independent polymer and a first water insoluble, pH independent polymer, and a permeable membrane coat containing a swellable, water soluble, pH independent polymer and a second water insoluble, pH independent polymer. The sustained release pharmaceutical dosage forms are free of osmagens and/or diluents and demonstrate similar release characteristics as compared to known sustained release formulations that contain osmagens and/or diluents. Additionally, the release profile for the sustained release pharmaceutical dosage forms is resistant to dose dumping in the presence of alcohol.

Placeholder for future cross-reference claim. This application claims the benefit of U.S. provisional application Ser. No. 63/400,108 filed Aug. 23, 2022 and 63/265,884 filed Dec. 22, 2021, the contents of which are incorporated herein in their entirety by reference.

### TECHNICAL FIELD

The present disclosure relates to sustained release pharmaceutical dosage forms of tofacitinib including a core and a permeable membrane coat surrounding the core. The core includes tofacitinib or a pharmaceutically acceptable salt thereof, at least one swellable, water soluble, pH independent polymer, and at least one first water insoluble, pH independent polymer. The permeable membrane coat includes at least one water soluble, pH independent polymer and at least one second water insoluble, pH independent polymer. The present disclosure also relates to methods of treating rheumatoid arthritis, psoriatic arthritis, ankylosing spondylitis, or ulcerative colitis by administering the sustained release formulations to a subject in need thereof. The sustained release formulations are free of osmagens and/or diluents and have desirable release characteristics and alcohol-resistance properties that protect against dose dumping in the presence of alcohol.

### BACKGROUND

Tofacitinib is a Janus kinase inhibitor, which is chemically designated as (3R,4R)-4-methyl-3-(methyl-7H-pyrrolo[2,3-d]pyrimidin-4-ylamino)-B-oxo-1-piperidinepropanenitrile. It is commonly prepared as a citrate salt in a 1:1 ratio. Tofacitinib is indicated for the treatment of adult patients with moderately to severely active rheumatoid arthritis and/or psoriatic arthritis who have had an inadequate response or intolerance to methotrexate, and for the treatment of adult patients with moderately to severely active ulcerative colitis who have had an inadequate response or who are intolerant to TNF blockers. It is marketed by Pfizer, Inc. as an extended release tablet under the brand name XELJANZ XR®.

The XELJANZ XR® tablets achieve sustained release through osmotic pump technology. The XELJANZ XR® tablet formulation is an osmotic pump consisting of a semi-permeable coating and a tofacitinib-containing core, and osmotic pressure is used to deliver the tofacitinib at controlled rate. The solute concentration gradient provides an osmotic force that drives the delivery of tofacitinib through an orifice (drilled hole) in the semi-permeable coating. The solute concentration gradient can be maintained constant when solute saturation is present in the tablet core. As the content of the core comes out through the orifice in the coating, the solute concentration, and thus the gradient and the osmotic force driving the drug release, declines. Oral sustained release formulations of tofacitinib achieved with osmotic pump technology are described, for example, in U.S. Pat. No. 9,937,181.

Although orifices in semi-permeable coatings may be achieved by mechanical means, mechanical methods capable of preparing the orifices at high manufacturing rates consistent with pharmaceutical manufacturing processes are not readily available. Even laser tablet drilling, which has become a technology of choice and is capable of leading to throughput rates of up to 100,000 tablets/hour, is expensive and requires an accepted-rejected system to monitor whether the laser-drilled hole meets accepted orifice-size specifications.

Accordingly, there is a need for additional or alternative sustained release formulations of tofacitinib that overcome the problems associated with the prior art and may be bioequivalent to XELJANZ XR®.

### SUMMARY

In light of the above, there is a need for additional or alternative sustained release formulations of tofacitinib. The present specification describes pharmaceutical dosage forms of tofacitinib that achieve sustained release of tofacitinib from a core whereby tofacitinib diffuses through a core and a permeable membrane coat that covers the core without the need for orifices or other similar modifications. The dosage form does not contain any osmagens and/or diluents (fillers). The dosage form also possesses desirable alcohol-resistance properties that protect against dose dumping of tofacitinib if the dosage form is in the presence of alcohol, for example when a subject ingests alcohol. The present specification also describes methods of treating rheumatoid arthritis, psoriatic arthritis, ankylosing spondylitis, or ulcerative colitis by administering the sustained release pharmaceutical dosage forms disclosed herein to a subject in need thereof.

In one aspect, disclosed is a sustained release dosage form that includes a core and a permeable membrane coat surrounding the core. The core includes tofacitinib or a pharmaceutically acceptable salt thereof, a swellable, water soluble, pH independent polymer having a molecular weight between about 150,000 to about 750,000, and a first water insoluble, pH independent polymer. The permeable membrane coat includes a water soluble, pH independent polymer and a second water insoluble, pH independent polymer, wherein the weight ratio of the water soluble, pH independent polymer to the second water insoluble, pH independent polymer in the permeable membrane coat is from about 0.4:1 to about 1.5:1.

Exemplary swellable, water soluble, pH independent polymers that can be included in the core can be chosen from polyethylene oxide; glyceryl fatty acid esters; hydrogenated castor oil; hydroxyethyl cellulose; hydroxypropyl cellulose; hydroxypropyl methylcellulose; ethylhydroxy ethylcellulose; methylethyl cellulose; carboxymethyl cellulose; carboxymethyl ethylcellulose; pullulan; polyvinyl pyrrolidone; polyvinyl alcohol; and polyvinyl acetate, and mixtures thereof.

Exemplary water soluble, pH independent polymers that can be included in the permeable membrane coat can be chosen from polyethylene oxide; glyceryl fatty acid esters; hydrogenated castor oil; hydroxyethyl cellulose; hydroxypropyl cellulose; hydroxypropyl methylcellulose; ethylhydroxy ethylcellulose; methylethyl cellulose; carboxymethyl cellulose; carboxymethyl ethylcellulose; pullulan; polyvinyl pyrrolidone; polyvinyl alcohol; polyvinyl acetate; ethyl acrylate; methyl methacrylate; and mixtures thereof.

Exemplary first water insoluble, pH independent polymers that can be included in the core can be chosen from copolymers of methacrylic acid or methacrylic acid esters; polyvinyl chloride; polyethylene; cellulose; cellulose derivatives; polyvinyl alcohol phthalate; polyvinyl acetate phthalate; polyvinylbutyl phthalate; polyvinyl acetate; polyvinyl acetate copolymers; crosslinked polyvinylpyrrolidone; carnauba wax; microcrystalline wax; triglycerides; and mixtures thereof.

Exemplary second water insoluble, pH independent polymers that can be included in the permeable membrane coat can be chosen from copolymers of methacrylic acid or methacrylic acid esters; polyvinyl chloride; polyethylene; cellulose; cellulose derivatives; polyvinyl alcohol phthalate; polyvinyl acetate phthalate; polyvinylbutyl phthalate; polyvinyl acetate; polyvinyl acetate copolymers; crosslinked polyvinylpyrrolidone; carnauba wax; microcrystalline wax; triglycerides; and mixtures thereof.

In one specific example, the first water insoluble, pH independent polymer and the second water insoluble, pH independent polymer are different from one another.

In another example, the sustained release dosage form is administered once daily.

In another example, the sustained release dosage form is in the form of a tablet.

In another example, the swellable, water soluble, pH independent polymer is present in the core in an amount of about 60% to about 75% by weight, based on the total weight of the core.

In another example, the first water insoluble, pH independent polymer is present in the core in an amount of from about 5% to about 20% by weight, based on the total weight of the core.

In another example, the weight ratio of the swellable, water soluble, pH independent polymer to the first water insoluble, pH independent polymer in the core is from about 3:1 to about 10:1.

In another example, the permeable membrane coat comprises about 3% to about 10% weight percent, based on the total weight of the dosage form.

In another example, the water soluble, pH independent polymer is present in the permeable membrane coat in an amount of from about 20% to about 40% weight percent, based on the total weight of the permeable membrane coat.

In another example, the weight ratio of the core in an uncoated state to the permeable membrane coat is about 15:1 to about 25:1, and the ratio of the swellable, water soluble, pH independent polymer to the water insoluble, pH independent polymer of the core is about 3:1 to about 10:1

In another example, the sustained release dosage form comprises from about 11 mg to about 22 mg of tofacitinib or an equivalent amount of tofacitinib in the form of a pharmaceutically acceptable salt. In certain instances, tofacitinib is in the form of a citrate salt.

In another example, the sustained release dosage form releases about 20% to about 40% of tofacitinib or an equivalent amount of a citrate salt thereof when the sustained release dosage form is subjected to a dissolution test at 50 rpm in 0.05M pH 6.8 potassium phosphate buffer at 37° C. for two hours.

In another example, the sustained release dosage form releases about 85% to about 100% of tofacitinib or an equivalent amount of a citrate salt thereof when the sustained release dosage form is subjected to the dissolution test for six hours.

In another example, the sustained release dosage form delivers tofacitinib or a pharmaceutically acceptable salt thereof to a subject by diffusion through the permeable membrane coat, and the permeable membrane coat covers the entire surface of the core.

In another example, the sustained release dosage form contains an enteric coating surrounding the permeable membrane coat.

In another example, the sustained release dosage form provides a zero-order release profile when administered to a subject.

In another example, the sustained release dosage form provides a release profile that is resistant to dose dumping in the presence of alcohol.

In another example, the sustained release dosage form is free of an osmagen, a diluent, or a combination thereof.

In another example, the core further includes a second swellable, water soluble pH independent polymer.

In another example, the second swellable, water soluble pH independent polymer has a molecular weight between about 150,000 to about 750,000.

In another example, the swellable, water soluble, pH independent polymer has a molecular weight between about 150,000 to about 250,000 and the second swellable, water soluble, pH independent polymer has a molecular weight between about 500,000 to about 750,000.

In another example, the swellable, water soluble, pH independent polymer has a viscosity between about 25 cP and about 500 cP, and the second swellable, water soluble, pH independent polymer has a viscosity between about 4,000 cP and about 10,000 cP.

In another example, the weight ratio of the swellable, water soluble, pH independent polymer to the second swellable, water soluble, pH independent polymer is from about 1:0.25 to about 1:2.5.

In another example, the core further includes an acidifier and is free of an osmagen, a diluent, or a combination thereof.

In another example, the acidifier is present in an amount of from about 2% to about 8% by weight, based on the total weight of the dosage form.

In another aspect, disclosed is a method of treating rheumatoid arthritis, psoriatic arthritis, ankylosing spondylitis, or ulcerative colitis by administering a sustained release pharmaceutical dosage form that includes a core and a permeable membrane coat surrounding the core to a subject in need thereof. The core includes tofacitinib or a pharmaceutically acceptable salt thereof, a swellable, water soluble, pH independent polymer having a molecular weight between about 150,000 to about 750,000, and a first water insoluble, pH independent polymer. The permeable membrane coat includes a water soluble, pH independent polymer and a second water insoluble, pH independent polymer, wherein the weight ratio of the water soluble, pH independent polymer to the second water insoluble, pH independent polymer in the permeable membrane coat is from about 0.4:1 to about 1.5:1.

In an exemplary method, the sustained release tofacitinib dosage form is free of an osmagen, a diluent, or a combination thereof.

In other exemplary methods, the sustained release dosage form comprises from about 11 mg to about 22 mg of tofacitinib or an equivalent amount of tofacitinib in the form of a pharmaceutically acceptable salt. In certain instances, tofacitinib is in the form of a citrate salt.

In further exemplary methods, the sustained release dosage form releases about 20% to about 40% of tofacitinib or an equivalent amount of a citrate salt thereof when the dosage form is subjected to a dissolution test at 50 or 150 rpm in 0.05M pH 6.8 potassium phosphate buffer at 37° C. for two hours, and the sustained release dosage form releases about 85% to about 100% of tofacitinib, or an equivalent amount of a citrate salt thereof, when the dosage form is subjected to the dissolution test for six hours.

Any one of the above aspects (or examples of those aspects) may be provided alone or in combination with any one or more of the examples of that aspect discussed above; e.g., the first aspect may be provided alone or in combination with any one or more of the examples of the first aspect discussed above; and the second aspect may be provided alone or in combination with any one or more of the examples of the second aspect discussed above; and so-forth.

Additional features and advantages will be set forth in the detailed description which follows, and in part will be readily apparent to those skilled in the art from that description or recognized by practicing the embodiments as described herein, including the detailed description which follows, the claims, as well as the appended figures.

It is to be understood that both the foregoing general description and the following detailed description are merely exemplary and are intended to provide an overview or framework to understanding the nature and character of the claims.

### DETAILED DESCRIPTION

The terminology as set forth herein is for description of the embodiments only and should not be construed as limiting the invention as a whole. Herein, when a range such as 5-25 (or 5 to 25) is given, this means preferably at least or more than 5 and, separately and independently, preferably not more than or less than 25. In an example, such a range defines independently 5 or more, and separately and independently, 25 or less. Moreover, all ranges disclosed herein are to be understood to encompass any and all values and sub-ranges between the stated values.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the endpoints of each of the ranges are significant both in relation to the other endpoint, and independently of the other endpoint.

Provided herein are sustained release pharmaceutical dosage forms of tofacitinib including a core and a permeable membrane coat surrounding the core. In certain instances, there is an enteric coating surrounding the permeable membrane coat. The core includes tofacitinib or a pharmaceutically acceptable salt thereof, at least one swellable, water soluble, pH independent polymer, and at least one first water insoluble, pH independent polymer. The permeable membrane coat includes at least one water soluble, pH independent polymer and at least one second water insoluble, pH independent polymer. The term “pharmaceutical dosage form,” as used herein, may include tablets, capsules, granules, and the like.

The term “tofacitinib,” as used herein, refers to tofacitinib free base or pharmaceutically acceptable salts thereof, in particular pharmaceutically acceptable acid addition salts including, for example, citrate, hydrochloride, hydrobromide, hydroiodide, nitrate, sulfate, bisulfate, phosphate, acetate, lactate, tartarate, succinate, malate, maleate, oxalate, fumarate, gluconate, saccharate, benzoate, methansulfonate, ethanesulfonate, benzenesulfonate, and the like. In specific instances, the pharmaceutically acceptable salt is the citrate salt.

The sustained release dosage forms described herein comprise from about 11 mg to about 22 mg of tofacitinib or an equivalent amount of tofacitinib in the form of a pharmaceutically acceptable salt. In one example, the dosage form includes about 11 mg tofacitinib. In another example, the dosage form includes about 22 mg tofacitinib.

The pharmaceutical dosage forms described herein may comprise from 10 mg to 24 mg of tofacitinib or an equivalent amount of tofacitinib in the form of a pharmaceutically acceptable salt. In one example, the dosage form includes a core and a permeable membrane coat surrounding the core. The core and the permeable membrane coat cooperate to sustain the release of tofacitinib. That is, the release characteristics of the core and the permeable membrane coat achieve, in combination, an in vitro drug release (dissolution) profile. In another example, the dosage form includes a core, a permeable membrane coat surrounding the core, and an enteric coating surround the core and the permeable membrane coat. The core, the permeable membrane coat, and the enteric coating cooperate to sustain the release of tofacitinib. That is, the release characteristics of the core, the permeable membrane coat, and the enteric coating achieve, in combination, an in vitro drug release (dissolution) profile. In some embodiments, the release profile of the dosage form can be equivalent to that of XELJANZ XR®. The weight ratio of the uncoated core to the permeable membrane coat is from about 5:1 to about 50:1, from about 10:1 to about 35:1, or from about 15:1 to about 25:1.

The core includes tofacitinib or a pharmaceutically acceptable salt thereof, at least one swellable, water soluble, pH independent polymer, and at least one water insoluble, pH independent polymer. The term “swellable, water soluble, pH independent polymer,” as used herein refers to polymers which imbibe water and form an aqueous-swollen gel or matrix that retains tofacitinib. The aqueous swollen matrix gradually swells in the environment of use, thereby controlling the release of tofacitinib. An acid or base, for example as those typically present in the human stomach or intestinal tract, is required to ionize the polymeric matrix sufficiently to cause erosion or dissolution. Swellable, water soluble, pH independent polymers include, but are not limited to, polyethylene oxide, in particular polyethylene oxide water soluble resins (such as those marketed under the name Polyox®, including Polyox WSR N80, Polyox 301, Polyox 303, Polyox 205, Polyox WSR Coagulant); glyceryl fatty acid esters, e.g., glyceryl behenate, glyceryl monostearate, glycerol distearate, glycerol monooleate, acetylated monoglycerides, tristearin, tripalmitin, cetyl esters wax, glyceryl palmitoste arate, and glyceryl behenate; hydrogenated castor oil; cellulose derivatives, e.g., hydroxyethyl cellulose, hydroxypropyl cellulose, hydroxypropyl methylcellulose, ethylhydroxy ethylcellulose, methylethyl cellulose, carboxymethyl cellulose, and carboxymethyl ethylcellulose; pullulan; polyvinyl pyrrolidone; polyvinyl alcohol; polyvinyl acetate; and mixtures of one or more of these polymers. In one embodiment, the swellable, water soluble, pH independent polymer is a polyethylene oxide. Ideally, the swellable, water soluble, pH independent polymer has a molecular weight of less than about 5,000,000, less than about 1,000,000, less than about 500,000, or less than about 250,000. In a specific embodiment, the swellable, water soluble, pH independent polymer has a molecular weight in a range of about 25,000 to about 5,000,000, about 50,000 to about 1,000,000, about 100,000 to about 800,000, about 200,000 to about 700,000, and about 400,000 to about 600,000. Preferably, the swellable, water soluble, pH independent polymer has a viscosity of less than about 20,000 cP, less than about 15,000 cP, or less than about 10,000 cP at room temperature (20° C. to 25° C.). In a specific embodiment, the swellable, water soluble, pH independent polymer has a viscosity in a range of about 5 cP to about 20,000 cP, about 25 cP to about 15,000 cP, or about 50 cP to about 10,000 cP at room temperature (20° C. to 25° C.).

In another embodiment, the uncoated core contains at least two swellable, water soluble, pH independent polymers. In a particular embodiment, the first swellable, water soluble pH independent polymer and the second swellable, water soluble pH independent polymer have different viscosities and/or molecular weights. In a specific example of this embodiment, the first swellable, water soluble pH independent polymer has a viscosity that is less than about 1,000 cP at room temperature (20° C. to 25° C.), and the second swellable, water soluble pH independent polymer has a viscosity that is greater than 1,000 cP at room temperature (20° C. to 25° C.). More specifically, the first swellable, water soluble pH independent polymer has a viscosity in a range of about 25 cP to about 1,000 cP at room temperature (20° C. to 25° C.), and the second swellable, water soluble pH independent polymer has a viscosity in a range of about 4,000 cP to about 10,000 cP at room temperature (20° C. to 25° C.). In another example of this embodiment, the first swellable, water soluble pH independent polymer has a molecular weight less than about 250,000, and the second swellable, water soluble pH independent polymer has a molecular weight greater than 250,000. More specifically, the first swellable, water soluble pH independent polymer has a molecular weight in a range of about 150,000 to about 250,000, and the second swellable, water soluble pH independent polymer has a molecular weight in a range of about 500,000 to about 750,000.

The weight ratio of the first, lower viscosity and/or lower molecular weight swellable, water soluble, pH independent polymer in the core to the second, higher viscosity and/or higher molecular weight, swellable, water soluble, pH independent polymer in the core in the pharmaceutical dosage form disclosed herein is from about 1:0.01 to about 1:25, from about 1:0.05 to 1:15, from about 1:0.1 to about 1:5, or from about 1:0.25 to about 1:2.5. In any event, the total amount of swellable, water soluble, pH independent polymer present in the uncoated core may be from about 40% to about 95%, about 50% to about 85%, or about 60% to about 75%, based on the total weight of the uncoated core.

The term “water insoluble, pH independent polymer,” as used herein refers to polymers which control the drug release by diffusion. The use of water insoluble, pH independent polymer in the core and in the permeable membrane coat imparts desirable alcohol-resistance properties to the dosage form that resist dose dumping of tofacitinib if the dosage form is in the presence of alcohol, for example when a subject ingests alcohol. Without being bound to theory, it is believed that the release profile of the dosage form is resistant to dose dumping in the presence of alcohol because the water insoluble, pH independent polymers in the permeable membrane coat and the core are also insoluble in alcohol. Suitable water insoluble, pH independent polymers include, but are not limited to, copolymers of methacrylic acid or methacrylic acid esters; polyvinyl chloride; polyethylene; cellulose and cellulose derivatives (e.g., ethylcellulose, cellulose acetate, cellulose acetate phthalate (CAP), hydroxypropyl methyl cellulose acetate succinate (HPMCAS), hydroxypropyl methylcellulose phthalate (HPMCP), and cellulose acetate succinate (CAS); polyvinyl polymers, e.g., polyvinyl alcohol phthalate, polyvinyl acetate phthalate, and polyvinylbutyl phthalate; polyvinyl acetate or polyvinyl acetate copolymers (including Kollicoat SR 30 D); crosslinked polyvinylpyrrolidone (also known as crospovidone); and fatty compounds, e.g., carnauba wax, microcrystalline wax, and triglycerides; and mixtures of one or more of these polymers. In one example, the water insoluble, pH independent polymer for use in the core is selected from among polyvinyl acetate, polyvinyl acetate copolymer, or mixtures thereof. The at least one water insoluble, pH independent polymer may be present in the uncoated core in an amount of about 1% to about 50%, about 5% to about 35%, about 5% to about 20%, or about 10% to about 20% by weight, based on the total weight of the uncoated core.

The weight ratio of the total weight of swellable, water soluble, pH independent polymer(s) in the core to the total weight of water insoluble, pH independent polymer(s) in the core provides a desirable release profile. The weight ratio of the total weight of swellable, water soluble, pH independent polymer(s) in the core to the total weight of water insoluble, pH independent polymer(s) in the core in the pharmaceutical dosage form disclosed herein is from about 1:1 to about 25:1, from about 2:1 to 15:1, from about 3:1 to about 10:1, or from about 3.5:1 to about 7:1.

The permeable membrane coat for use in the pharmaceutical dosage form includes at least one water soluble, pH independent polymer and at least one water insoluble pH independent polymer. The term “permeable membrane coat,” as used herein, describes a membrane coating that covers the entire outer surface of the core through which water passes to dissolve drugs or solutes in the core, and the dissolved drugs or solutes diffuse to the environment external to the dosage form.

In contrast, the term “semipermeable membrane coat,” as used herein, includes a membrane through which water readily diffuses through the means of a membrane, but solutes dissolved in water typically cannot readily diffuse through the membrane. The osmotic tablet currently marketed as XELJANZ XR® contains a semipermeable membrane which includes one or more laser drilled orifices (openings). As the XELJANZ XR® tablet passes through the body, the osmotic pressure of water entering the tablet through the semipermeable membrane coat pushes the drug through the opening(s) in the semipermeable membrane. Thus, unlike the semipermeable membrane coat used in existing commercially available formulations, the permeable membrane coat of the pharmaceutical dosage forms described herein provides a zero-order release without the need for orifices (openings) in the membrane coat.

The permeable membrane coat of the pharmaceutical dosage form disclosed herein is present in about 1% to about 20%, about 2% to about 15%, or about 3% to about 10% by weight, based on the total weight of the dosage form.

The term “water soluble, pH independent polymer,” as used herein refers to polymers that are dissolvable in water and that cause sufficient dissolution of the polymeric coating. When contacted with an aqueous environment, the water soluble, pH independent polymer gradually dissolves in the environment of use, thereby permitting water to pass through the permeable membrane coat and into the core. Thus, the rate at which water soluble, pH independent polymer in the permeable membrane coat dissolves can affect the release of tofacitinib. Water soluble, pH independent polymers for use in the permeable membrane coat include, but are not limited to polyethylene oxides; glyceryl fatty acid esters, e.g., glyceryl behenate, glyceryl monostearate, glycerol distearate, glycerol monooleate, acetylated monoglycerides, tristearin, tripalmitin, cetyl esters wax, glyceryl palmitostearate, and glyceryl behenate; hydrogenated castor oil; cellulose derivatives, e.g., hydroxyethyl cellulose, hydroxypropyl cellulose, hydroxypropyl methylcellulose, ethylhydroxy ethylcellulose, methylethyl cellulose, carboxymethyl cellulose, and carboxymethyl ethylcellulose; pullulan; polyvinyl pyrrolidone (povidone); polyvinyl alcohol; polyvinyl acetate, acrylate derivatives, e.g., ethyl acrylate and methyl methacrylate, and mixtures thereof. In a specific embodiment, the water soluble, pH independent polymer for use in the permeable membrane coat is a cellulose derivative, an acrylate derivative, or mixtures thereof. The at least one water soluble, pH independent polymer may be present in the permeable membrane coat in an amount of about 5% to about 60%, about 15% to about 50%, or about 20% to about 40% by weight, based on the total weight of the permeable membrane coat.

Water insoluble, pH independent polymers for use in the permeable membrane coat may be selected from the group of polymers detailed above as suitable for use as water insoluble, pH independent polymers in the core. In one embodiment, the at least one water insoluble, pH independent polymer in the permeable membrane coat and the at least one water insoluble, pH independent polymer in the core are the same. In another embodiment, the at least one water insoluble, pH independent polymer in the permeable membrane coat and the at least one water insoluble, pH independent polymer in the core are selected independently of each other and can be different from one another. In a specific embodiment, the at least one water insoluble, pH independent polymer for use in the permeable membrane coat is polyvinyl acetate. The at least one water insoluble, pH independent polymer may be present in the permeable membrane coat in an amount of about 5% to about 65%, about 15% to about 55%, or about 25% to about 45% by weight, based on the total weight of the permeable membrane coat.

The weight ratio of the total weight of water soluble, pH independent polymer(s) in the permeable membrane coat to the total weight of water insoluble, pH independent polymer(s) in the permeable membrane coat also contributes to obtaining a desirable release profile. The weight ratio of the total weight of water soluble, pH independent polymer(s) in the permeable membrane coat to the total weight of the water insoluble, pH independent polymer(s) in the permeable membrane coat in pharmaceutical dosage forms disclosed herein is from about 0.4:1 to about 1.5:1, from about 0.5:1 to about 1.3:1, from about 0.5:1 to about 1.2:1, or from about 0.6:1 to about 1.2:1.

The core and the permeable membrane coat of the pharmaceutical dosage forms may further include one or more pharmaceutically acceptable excipients. As used herein, term “pharmaceutically acceptable excipients” includes any physiologically inert additives that are routinely used in pharmaceutical dosage forms. Pharmaceutically acceptable excipients may be selected from the group including, for example, binders, glidants, lubricants, plasticizers, surfactants, opacifiers, disintegrants, and acidifiers among others.

Suitable glidants include, but are not limited to, magnesium stearate, stearic acid, calcium stearate, colloidal silicon dioxide, starch, talc, and combinations thereof.

Suitable lubricants include, but are not limited to, magnesium stearate, talc, and silica and combinations thereof.

Suitable plasticizers include, but are not limited to, triethyl citrate, dibutyl sebacate, acetylated triacetin, tributyl citrate, glyceryl tributyrate, monoglyceride, rapeseed oil, olive oil, sesame oil, acetyl tributyl citrate, acetyl triethyl citrate, glycerin, sorbitol, diethyloxalate, diethyl phthalate, diethyl malate, diethyl fumarate, dibutyl succinate, diethyl malonate, dioctyl phthalate, and combinations thereof.

Suitable surfactants include, but are not limited to, sorbitan monolaurate, sorbitan trioleate, polyoxyethylene sorbital, sorbitan tristearate, polyoxyethylene sorbital hexastearate, ethylene glycol fatty acid ester, propylene glycol fatty acid ester, propylene glycol monostearate, glycerol monostearate, sorbitan monooleate, and combinations thereof.

Suitable opacifiers include, but are not limited to, titanium dioxide.

Suitable disintegrants include, but are not limited to, croscarmellose sodium, hydroxypropyl cellulose (L-HPC), crospovidone, carboxymethyl cellulose sodium, carboxymethyl cellulose calcium, sodium starch glycolate, gums, alginic acid or alginates, pregelatinized starch, corn starch, modified starch, carboxymethyl starch, polyacrylates, and combinations thereof.

In one embodiment, the dosage forms described herein are free of osmagens and/or diluents, or contain non-effective or non-detectable amounts thereof. Osmagens are osmotically effective solutes. Typical classes of suitable osmagens (also known as osmagents) include water-soluble salts, sugars, organic acids, and other low-molecule-weight organic compounds that are capable of imbibing water to thereby establish an osmotic pressure gradient across the barrier of the surrounding coating. Salts typically used as osmagens include magnesium sulfate, magnesium chloride, calcium chloride, sodium chloride, lithium chloride, potassium sulfate, sodium carbonate, sodium sulfite, lithium sulfate, potassium chloride, and sodium sulfate. Conventionally, chloride salts such as sodium chloride are utilized as osmagens. Because the pharmaceutical dosage forms disclosed herein achieve a sustained release profile by diffusing tofacitinib through a permeable membrane, the use of an osmagen to maintain a solute concentration for driving drug release through a semipermeable membrane is unnecessary.

One or more acidifiers may be added to the core to adjust the pH of the microenvironment through which the drug is released. A suitable acidifier includes, but is not limited to, citric acid monohydrate. It is believed that acidifiers included in the formulations disclosed herein (i.e., citric acid monohydrate), do not function as an osmagen in that they are not capable of imbibing water to the degree necessary to establish an osmotic pressure gradient across the barrier of the coating due to the presence of water molecules (as hydrates) in their molecular structure. The acidifier(s) may be present in an amount of from about 0% to about 25%, about 0.5% to about 15%, about 1% to about 10%, or about 2% to about 8% by weight of the dosage form.

Diluents (or fillers) are often incorporated into solid oral dosage forms in order to increase the bulk volume of the dosage form and achieve content uniformity. Typical diluents include lactose, e.g., directly compressible lactose (Pharmatose® DCL11), lactose monohydrate, lactose anhydrous, and spray dried lactose; microcrystalline cellulose, e.g., microcrystalline PH 112, microcrystalline PH 101, and microcrystalline PH 102; sugar alcohols, e.g., sorbitol, erythritol, xylitol, and mannitol; sugars, e.g., sucrose, DiPac® (a directly compressible, co-crystallized sugar consisting of 97% sucrose and 3% maltodextrin), and starch, e.g., pregelatinized starch. The incorporation of a swellable, water soluble pH independent polymer and a water insoluble, pH independent polymer in the core of the dosage forms advantageously eliminates the need for a diluent.

The sustained release pharmaceutical dosage forms disclosed herein may be made by conventional tableting and coating techniques. In one embodiment, the core is prepared by any convenient tableting technique, such as wet granulation and/or direct compression. In one specific method, tofacitinib, the at least one swellable, water soluble, pH independent polymer, the at least one water insoluble, pH independent polymer, and, optionally, one or more pharmaceutically acceptable excipients are blended and wet granulated. The granules are optionally mixed with additional pharmaceutically acceptable excipients, such as tableting lubricants, and are compressed to form cores.

The permeable membrane coat may be prepared by any conventional coating technique, such as spraying or dipping. In a specific method, previously prepared cores are added to a pan and are spray coated with a dispersion of the permeable membrane coat composition including at least one least one water soluble, pH independent polymer and at least one water insoluble pH independent polymer and, optionally, one or more pharmaceutically acceptable excipients. It should be noted that the dispersion is not itself a solution, though the water soluble, pH independent polymer may be included in the dispersion as a solution. The pan containing the cores is rotated as the dispersion is sprayed in order to ensure that the entirety of the cores are coated, preferably uniformly, with the dispersion. As the cores are sprayed with the dispersion, the rotating pan is simultaneously exposed to hot, dry air. Thus, the permeable membrane coat is simultaneously applied via spraying onto the cores and dried. The coated cores are weighed to ensure that the desired amount of dispersion has been applied.

Optionally, the coated cores may be additionally coated with a film coating, such as those provided under the trade name Opadry®.

In one or more embodiments, the cores coated with the permeable membrane may be further coated with an enteric coating. The enteric coating may be present in an amount of from about 0% to about 5%, about 0.5% to about 4%, about 1% to about 3.5%, or about 2% to about 3% by weight of the dosage form. The enteric coating contains one or more polymer components.

Example polymer components can include methacrylic acid copolymer (e.g., poly(methacrylic acid-co-methyl methacrylate), poly(methacrylic acid-co-ethyl methacrylate)), hydroxypropylmethylcellulose phthalate (hereinafter to be also referred to as hypromellose phthalate), hydroxypropylmethylcellulose acetate succinate (hereinafter to be also referred to as hypromellose acetate succinate), cellulose acetate phthalate, polyvinyl acetate phthalate, carboxymethylethylcellulose, shellac and the like can be mentioned. A a methacrylic acid copolymer can be constituted of methacrylic acid and one or more kinds of acrylate monomers, for example, methylacrylate, methylmethacrylate and ethylacrylate. Example methacrylic acid copolymers include Eudragit® L 30 D-55, L 100-55, L 100, L 12.5, S 100, S 12.5, and FS 30 D.

In one example, a methacrylic acid copolymer is the sole polymer component in the enteric coating. The polymer component of the enteric coating can aid in reducing the food effect on the dosage form. For example, the outer enteric coating can delay the release of tofacitinib until the dosage form has passed through the stomach and into the small intestine. In one or more embodiments, the enteric coating is designed to dissolve at a pH greater than 5.0, preferably at a pH of 6.0 or higher and most preferably a pH of 6.5 or higher.

The one or more polymer components of the enteric coating may be present in an amount of from about 0.5% to about 3%, or about 1% to about 2.5% by weight of the dosage form.

The enteric coating of the pharmaceutical dosage forms, when used, may further include one or more pharmaceutically acceptable excipients as defined above. For instance, pharmaceutically acceptable excipients may be selected from the group including, for example, binders, glidants, lubricants, plasticizers, surfactants, opacifiers, disintegrants, and acidifiers among others.

Suitable glidants include, but are not limited to, magnesium stearate, stearic acid, calcium stearate, colloidal silicon dioxide, starch, talc, and combinations thereof.

Suitable lubricants include, but are not limited to, magnesium stearate, talc, and silica and combinations thereof.

Suitable plasticizers include, but are not limited to, triethyl citrate, dibutyl sebacate, acetylated triacetin, tributyl citrate, glyceryl tributyrate, monoglyceride, rapeseed oil, olive oil, sesame oil, acetyl tributyl citrate, acetyl triethyl citrate, glycerin, sorbitol, diethyloxalate, diethyl phthalate, diethyl malate, diethyl fumarate, dibutyl succinate, diethyl malonate, dioctyl phthalate, and combinations thereof.

The enteric coating may be applied to the permeable membrane coat on the core by any conventional coating technique, such as spraying or dipping, followed by drying. To prepare a solution of dispersion for applying the enteric coating, the polymer component and excipients, such as glidants, lubricants, plasticizers and combinations thereof, can be added to a vessel for mixing with a solvent. The solution or dispersion can be continually mixed during a coating process for applying the enteric coat to the dosage form.

The sustained release pharmaceutical dosage forms of the present disclosure are suitable for oral administration, for example, to a mammal as an immunosuppressive agent for organ transplants or xenotransplantation, or to treat rheumatoid arthritis, psoriatic arthritis, ankylosing spondylitis, lupus, multiple sclerosis, Type I diabetes and complications from diabetes, cancer, asthma, atopic dermatitis, autoimmune thyroid disorders, juvenile idiopathic arthritis, Crohn's disease, Alzheimer's disease, leukemia, ulcerative colitis, and other indications where immunosuppression would be desirable. In a specific method, the sustained release pharmaceutical dosage form are suitable for oral administration to a mammal to treat rheumatoid arthritis, psoriatic arthritis, ankylosing spondylitis or ulcerative colitis. In one example, the mammal is a human. Example methods include administering the sustained release pharmaceutical dosage form to a human subject in need thereof in an amount effective to treat rheumatoid arthritis, psoriatic arthritis, ankylosing spondylitis, or ulcerative colitis. The sustained release dosage form may be administered, for example, once a day, twice a day, or as needed for a period of time necessary to treat rheumatoid arthritis, psoriatic arthritis, ankylosing spondylitis, or ulcerative colitis. In one example, the sustained release dosage forms described herein are administered once a day, such that the dosage form is characterized as once daily.

Sustained release dosage forms of tofacitinib according to the present disclosure will be illustrated in the following non-limiting examples. The examples should not, however, be viewed as limiting the scope of the invention. The claims will serve to define the invention.

### EXAMPLES

### Example 1

11 mg and 22 mg formulations of the reference drug, XELJANZ XR®, were subjected to a dissolution test conducted in a standard USP rotating paddle apparatus as disclosed in United States Pharmacopoeia (USP) Dissolution Test Chapter 711, Apparatus 2. Paddles were rotated at 50 rpm and the dosage form was added to 900 mL of 0.05M pH 6.8 potassium phosphate buffer at 37° C. At appropriate times following initiation of the dissolution test (i.e. insertion of the dosage form into the apparatus), filtered aliquots (typically 1.5 mL) from the test medium were analyzed for tofacitinib by high performance liquid chromatography (HPLC). Dissolution results are reported below in Table 1 as the percent of the total dose of tofacitinib tested versus time.

TABLE 1

| Time | 11 mg tablet | 22 mg tablet |
|---|---|---|
| (hr.) | % drug dissolved | % drug dissolved |
| 0 | 0 | 0.00 |
| 1 | 2.7 | 5.06 |
| 1.5 | 12.1 | 18.50 |
| 2 | 24.7 | 35.30 |
| 2.5 | 39.5 | 51.01 |
| 3 | 53.3 | 62.54 |
| 4 | 71.6 | 75.18 |
| 6 | 85.3 | 87.54 |
| 8 | 91.9 | 92.36 |

### Example 2

In order to match the in vitro drug release profile for reference product based on osmotic release technology with a formulation based on diffusion through a permeable coat, a core formulation with a relatively faster drug release profile than the reference product was sought. Ten samples were prepared by blending 35.54 mg of tofacitinib citrate (equivalent to 22 mg tofacitinib), a swellable, water soluble, pH independent polymer, a water insoluble, pH independent polymer, or mixtures thereof, to form final blends. The final blends were combined with 2.0 mg of magnesium stearate and, in some instances, 4.0 mg colloidal silicone dioxide and then compressed to form cores. The contents of the ten samples are set forth in Table 2, below.

TABLE 2

| | Example number | | | | | | | | | |
|---|---|---|---|---|---|---|---|---|---|---|
| | 1 | 2 | 3 | 4 | 5 | 6 | 7 | 8 | 9 | 10 |
| Ingredient | Amount (mg) per core | | | | | | | | | |
| Tofacitinib | 35.54 | 35.54 | 35.54 | 35.54 | 35.54 | 35.54 | 35.54 | 35.54 | 35.54 | 35.54 |
| citrate | | | | | | | | | | |
| Kollidon | 162.46 | 81.23 | 138.12 | 109.72 | 79.23 | 79.23 | | 35.4 | 35.4 | 35.4 |
| SR | | | | | | | | | | |
| Polyox | | 81.23 | 24.34 | 52.74 | | | 162.46 | | | |
| WSR N 80 | | | | | | | | | | |
| LEO | | | | | | | | | | |
| Polyox 303 | | | | | | | | 127.06 | | |
| Polyox 205 | | | | | | | | | 127.06 | |
| Polyox | | | | | | | | | | 127.06 |
| coagulant | | | | | | | | | | |
| HPMC | | | | | 79.23 | | | | | |
| K4M | | | | | | | | | | |
| Premium | | | | | | | | | | |
| Xanthan | | | | | | 79.23 | | | | |
| Gum | | | | | | | | | | |
| Colloidal | | | | | 4.0 | 4.0 | | 4.0 | 4.0 | 4.0 |
| silicone | | | | | | | | | | |
| dioxide | | | | | | | | | | |
| Magnesium | 2.0 | 2.0 | 2.0 | 2.0 | 2.0 | 2.0 | 2.0 | 2.0 | 2.0 | 2.0 |
| stearate | | | | | | | | | | |
| Total Core | 200 | 200 | 200 | 200 | 200 | 200 | 200 | 204 | 204 | 204 |
| tablet | | | | | | | | | | |

The samples were subjected to the dissolution test detailed above in Example 1. The results obtained are set forth below in Table 3.

TABLE 3

| | Sample number | | | | | | | | | |
|---|---|---|---|---|---|---|---|---|---|---|
| Time | (% drug dissolved) | | | | | | | | | |
| (hours) | 1 | 2 | 3 | 4 | 5 | 6 | 7 | 8 | 9 | 10 |
| 0 | 0 | 0 | 0 | 0 | 0 | 0 | 0 | 0 | 0 | 0 |
| 1 | 7.5 | 15.2 | 9.3 | 12.5 | 9.3 | 8.8 | 42.6 | 9.8 | 13.9 | 9.2 |
| 1.5 | 9.1 | 20.0 | 11.7 | 15.9 | 12.3 | 12.2 | 63.8 | 13.1 | 20.1 | 12.2 |
| 2 | 10.4 | 24.0 | 13.6 | 18.8 | 15.2 | 15.7 | 80.4 | 16.3 | 26.2 | 15.3 |
| 2.5 | 11.6 | 27.5 | 15.3 | 21.4 | 17.8 | 18.7 | 92.2 | 19.5 | 32.0 | 18.2 |
| 3 | 12.7 | 30.6 | 16.9 | 23.7 | 20.2 | 21.7 | 99.7 | 22.5 | 37.4 | 21.1 |
| 4 | 14.4 | 36.1 | 19.5 | 27.8 | 24.6 | 27.4 | 101.7 | 28.2 | 47.4 | 26.6 |
| 6 | 17.2 | 45.0 | 24.0 | 34.2 | 31.1 | 36.5 | 102.1 | 37.1 | 64.9 | 35.6 |

None of the ten samples evidenced acceptable in vitro drug release compared to the reference drug dissolution profile. In most instances, the release profiles for the samples were very slow compared to the reference drug release profile. In contrast, Sample 7, which did not contain a water insoluble, pH independent polymer, evidenced an excessively fast release profile as compared to the reference drug. Thus, Table 3 indicates that water insoluble, pH independent polymer significantly retards drug release, though the degree depends on the amount of water insoluble, pH independent polymer and the character and amount of swellable, water soluble, pH independent polymer.

### Example 3

Following the results of Table 2 above, the effect of the amounts of swellable, water soluble, pH independent polymer and water insoluble, pH independent polymer in the core were investigated. Six experimental cores were prepared by blending 35.54 mg of tofacitinib citrate (equivalent to 22 mg tofacitinib), a swellable, water soluble, pH independent polymer (Polyox WSR N 80 LEO) and a water insoluble, pH independent polymer (Kollidon SR) to form final blends. The final blends were combined with 2.0 mg of magnesium stearate and then compressed to form cores. In each of the six samples, the amount of the swellable, water soluble, pH independent polymer and the water insoluble, pH independent polymer were varied. The contents of the six samples are set forth in Table 4, below.

TABLE 4

| | Sample number | | | | | |
|---|---|---|---|---|---|---|
| | 1 | 2 | 3 | 4 | 5 | 6 |
| Ingredients | Amount (mg) per core | | | | | |
| Tofacitinib citrate | 35.54 | 35.54 | 35.54 | 35.54 | 35.54 | 35.54 |
| Kollidon SR | 20.3 | 40.62 | 60.9 | 38.46 | 35.4 | 32.46 |
| Polyox WSR N 80 | 142.16 | 121.84 | 101.26 | 124.0 | 127.0 | 130.0 |
| LEO | | | | | | |
| Magnesium | 2.0 | 2.0 | 2.0 | 2.0 | 2.0 | 2.0 |
| stearate | | | | | | |
| Total Core | 200 | 200 | 200 | 200 | 200 | 200 |
| tablet | | | | | | |

The samples were subjected to the dissolution test detailed above in Example 1. The results are set forth below in Table 5.

TABLE 5

| | |
|---|---|
| | Sample number |
| Time | (% drug dissolved) |

| (hours) | 1 | 2 | 3 | 4 | 5 | 6 |
|---|---|---|---|---|---|---|
| 0 | 0 | 0 | 0 | 0 | 0 | 0 |
| 1 | 40.6 | 22.9 | 18.9 | 23.2 | 26.6 | 30.0 |
| 1.5 | 59.8 | 31.8 | 24.8 | 32.6 | 38.4 | 43.3 |
| 2 | 74.0 | 40.3 | 30.0 | 40.6 | 49.3 | 54.4 |
| 2.5 | 84.7 | 44.8 | 34.6 | 48.0 | 57.9 | 63.4 |
| 3 | 93.1 | 55.4 | 39.0 | 55.0 | 65.3 | 71.2 |
| 4 | 100.4 | 67.5 | 46.1 | 66.5 | 78.2 | 84.1 |
| 6 | 101.1 | 86.0 | 57.8 | 82.9 | 91.6 | 95.7 |
| 8 | 101.1 | 94.4 | 66.4 | 92.1 | 96.2 | 96.9 |

Sample 3, which had the lowest weight ratio of swellable, water soluble, pH independent polymer to water insoluble, pH independent polymer in the core (1.66:1), exhibited a dissolution profile that was slower than the dissolution profile for the reference drug product. Samples 2 and 4, which had weight ratios of swellable, water soluble, pH independent polymer to water insoluble, pH independent polymer of 3:1 and 3.22:1, respectively, exhibited a release profile that was similar to the release profile for the reference drug product at the 2, 2.5, 6 and 8 hour time points, but faster at the other time points. Samples 5 and 6, which had weight ratios of swellable, water soluble, pH independent polymer to water insoluble, pH independent polymer of 3.59:1 and 4:1, respectively, exhibited faster dissolution release profiles than that for the reference drug product, though not as fast as Sample 1. Sample 1, which had the highest weight ratio of swellable, water soluble, pH independent polymer to water insoluble, pH independent polymer in the core (7:1), exhibited a dissolution profile that was very fast compared to the dissolution profile for the reference drug product. Thus, Table 5 indicates that cores containing a weight ratio of swellable, water soluble, pH independent polymer to water insoluble, pH independent polymer between about 3:1 and about 7:1 demonstrated a desirable release profile for use in a diffusion-based sustained release dosage form. The desirable weight ratio of swellable, water soluble, pH independent polymer to water insoluble, pH independent polymer in core tablet formulation is further optimized with application of permeable film coating on core tablet.

### Example 4

Permeable membrane coatings intended to retard drug release from the core with a water insoluble, pH independent polymer were explored. Cores were prepared by blending 35.54 mg of tofacitinib citrate (equivalent to 22 mg tofacitinib), 127.06 mg of Polyox WSR N 80 LEO (a swellable, water soluble, pH independent polymer) and 35.4 mg of Kollidon SR (a water insoluble, pH independent polymer) to form final blends. The final blends were combined with 2.0 mg of magnesium stearate and 4.0 mg of colloidal silicon dioxide, and then compressed to form cores. The cores were divided into three experimental samples intended to receive different amounts of a permeable membrane coating, as indicated below in Table 6. Each sample was added to a perforated tablet coating pan, and spray coated with a dispersion of a coating including Kollicoat SR 30 D (a water insoluble, pH independent polymer) with plasticizer, glidant and opacifier. The pan was rotated and exposed to hot, dry air to dry the cores. The coated cores were weighed to ensure that the desired amount of coating had been applied. The contents of each sample are set forth below in Table 6. Each coated sample was subjected to the dissolution test detailed above in Example 1.

TABLE 6

| Sample number | 1 | 2 | 3 |
|---|---|---|---|
| Ingredients | Amount (mg) per tablet | | |
| Tofacitinib citrate | 35.54 | 35.54 | 35.54 |
| Kollidon SR | 35.4 | 35.4 | 35.4 |
| Polyox WSR N 80 LEO | 127.06 | 127.06 | 127.06 |
| Colloidal silicon dioxide | 4.0 | 4.0 | 4.0 |
| Magnesium stearate | 2.0 | 2.0 | 2.0 |
| Total Core weight | 204 | 204 | 204 |
| Permeable Membrane Coat | w/w % of coating by core weight | | |
| Kollicoat SR 30 D with | 3.6% | 6.4% | 7.9% |
| plasticizer, glidant and | w/w | w/w | w/w. |
| opacifier | | | |

For each of the samples in Table 6, no in vitro drug release observed. Thus, Table 7 indicates that a coating based only on a water insoluble, pH independent polymer does not release any drug, and therefore is not ideal.

### Example 5

Coating formulations including water soluble, pH independent polymers were also evaluated. Cores were prepared by blending 35.54 mg of tofacitinib citrate (equivalent to 22 mg tofacitinib), 137.00 mg of Polyox WSR N 80 LEO (a swellable, water soluble, pH independent polymer) and 25.46 mg of Kollidon SR (a water insoluble, pH independent polymer) to form final blends. The final blends were combined with 2.0 mg of magnesium stearate and 4.0 mg of colloidal silicon dioxide, and then compressed to form cores. The cores were divided into four samples. Each sample was coated with a different coating in the manner detailed above for Example 4. Each coating contained a water soluble, pH independent polymer selected from among HPMC E5, Eudragit NM 30 D, and Methylcellulose A 15 LV, Triethyl citrate as a plasticizer, titanium dioxide as an opacifier, talc as a glidant, and red color as a colorant. In some instances, stearic acid was added to the coating as an additional release controlling agent. A water insoluble, pH independent polymer (Kollicoat SR 30 D) was also added to the coating formulation containing HPMC E5 to observe the effect of a combination of a water insoluble, pH independent polymer and a water soluble, pH independent polymer on the release profile. The content of the different coatings are detailed below in Table 7.

TABLE 7

| Sample No. | 1 | 2 | 3 | 4 |
|---|---|---|---|---|
| Ingredients | mg/unit | | | |
| Tofacitinib citrate | 35.54 | 35.54 | 35.54 | 35.54 |
| Polyox N 80 | 137.00 | 137.00 | 137.00 | 137.00 |
| Kollidon SR | 25.46 | 25.46 | 25.46 | 25.46 |
| Colloidal silicon dioxide | 4 | 4 | 4 | 4 |
| Magnesium stearate | 2 | 2 | 2 | 2 |
| Sub-total (Core) | 204 | 204 | 204 | 204 |
| Kollicoat SR 30 D | — | — | — | 4.65 |
| HPMC E 5 | — | — | — | 1.40 |
| Eudragit NM 30 D | — | — | 3.06 | — |
| Methylcellulose A 15 LV | 6.96 | 5.88 | — | — |
| Stearic acid | 2.32 | 1.96 | — | — |
| Triethyl citrate | 0.58 | 0.49 | — | 0.47 |
| Titanium dioxide | 0.58 | 0.49 | — | 0.47 |
| Talc | 1.16 | 0.98 | 3.06 | 2.33 |
| Red color | — | — | — | 0.20 |
| Purified water | Q.S. | Q.S. | Q.S. | Q.S. |
| Total | 215.6 | 213.8 | 210.12 | 213.52 |

The four coated samples were subjected to the dissolution test detailed above in Example 1. The results are set forth below in Table 8.

TABLE 8

| | Sample No. | | | |
|---|---|---|---|---|
| | (% drug dissolved) | | | |
| Time (hours) | 1 | 2 | 3 | 4 |
| 0 | 0 | 0.0 | 0 | 0 |
| 1 | 6.2 | 10.6 | 0.0 | 1.9 |
| 1.5 | 13.2 | 18.6 | 0.0 | 6.2 |
| 2 | 20.6 | 26.4 | 0.0 | 12.7 |
| 2.5 | 28.2 | 34.1 | 0.2 | 20.4 |
| 3 | 35.1 | 41.5 | 0.3 | 28.6 |
| 4 | 48.2 | 56.9 | 0.9 | 43.6 |
| 6 | 72.0 | 78.8 | 4.6 | 64.0 |
| 8 | 92.1 | 84.9 | 11.7 | 76.1 |

Each of the sample coatings containing a water soluble, pH independent polymer exhibited a release profile that was slower than the reference drug release profile. Sample 3 containing Eudragit NM 30 D, a combination of ethyl acrylate and methyl methacrylate, exhibited a dissolution profile that was very slow compared to the dissolution profile for the reference drug product. Samples 1 and 2 contained the same coating excipients, except that Sample 2 contained approximately 15.3% less of each coating excipients than Sample 1, in other words, contains less coating weight gain. Both release profiles were slower than the release profile for the reference drug product, but the release profile for Sample 1 was slower than the dissolution profile for Sample 2 due to difference in coating weight gain. On the other hand, Sample 3 contained only water insoluble, pH independent polymer without any water soluble, pH independent polymer, which resulted in slowest drug release. Thus, Table 8 indicates that a higher amount of water soluble, pH independent polymer in the coating resulted in a faster release profile. Sample 4 containing a combination of water soluble, pH independent polymer and water insoluble, pH independent polymer in a weight ratio of 1:3.32 exhibited a release profile that was slower than the release profiles for the reference drug and Samples 1 and 2. Accordingly, Table 8 also indicates that the combination of a water soluble, pH independent polymer and a water insoluble, pH independent polymer retards drug release, though the degree of release is dependent on the character and amount of water soluble, pH independent polymer and water insoluble, pH independent polymer.

### Example 6

Cores were prepared by blending 35.54 mg of tofacitinib citrate (equivalent to 22 mg tofacitinib), Polyox WSR N 80 LEO (a swellable, water soluble, pH independent polymer) and Kollidon SR (a water insoluble, pH independent polymer) to form final blends. The final blends were combined with 4.0 mg of magnesium stearate and 4.0 mg of colloidal silicon dioxide, and then compressed to form cores. The cores were coated with one of two different permeable membrane coatings in the manner detailed above for Table 6. Each coating contained different amounts of HPMC E5 and Kollicoat SR 30 D. The coating formulations also contained triethyl citrate as a plasticizer, titanium dioxide as an opacifier, talc as a glidant. A film coating, Opadry II Yellow, was applied to the coated cores after drying. The content of the cores and coatings is detailed below in Table 9.

TABLE 9

| | | |
|---|---|---|
| Sample No. | 1 | 2 |
| Ingredients | mg/unit | mg/unit |

Core

| | | |
|---|---|---|
| Tofacitinib Citrate | 35.54 | 35.54 |
| Polyox N 80 | 145.46 | 140.00 |
| Kollidone SR | 15.00 | 20.46 |
| Colloidal silicon dioxide | 4 | 4 |
| Magnesium stearate | 4 | 4 |
| Sub-total (Core) | 204 | 204 |

Permeable Membrane Coating

| | | |
|---|---|---|
| Kollicoat SR 30 D | 4.284 | 4.08 |
| HPMC E 5 | 4.998 | 2.55 |
| Triethyl citrate | 0.714 | 0.51 |
| Titanium dioxide | 0.714 | 0.51 |
| Talc | 3.570 | 2.55 |
| Purified water | Q.S. | Q.S. |
| Sub-total (Core + Coating) | 218.28 | 214.2 |
| Opadry II Yellow | 6.55 | 6.43 |
| Total | 224.83 | 220.63 |

Both samples were subjected to the dissolution test detailed above for Example 1. The results obtained are set forth below in Table 10.

TABLE 10

| | Sample No. | |
|---|---|---|
| | (% drug dissolved) | |
| Time (hours) | 1 | 2 |
| 0 | 0 | 0 |
| 1 | 15.1 | 11.9 |
| 1.5 | 28.5 | 22.0 |
| 2 | 41.4 | 32.0 |
| 2.5 | 53.7 | 41.5 |
| 3 | 64.8 | 50.8 |
| 4 | 83.3 | 67.5 |
| 6 | 98.5 | 86.2 |
| 8 | 101.3 | 90.0 |

Core formulation of Sample 1 and Sample 2 contained a weight ratio of swellable, water soluble, pH independent polymer to water insoluble, pH independent polymer of 9.7:1 and 6.8:1, respectively. Sample 1, which contained a permeable membrane coat having a weight ratio of water soluble, pH independent polymer:water insoluble, pH independent polymer of about 1.167:1, resulted in a release profile that was slightly faster compared to the release profile for the reference drug product. In contrast, Sample 2, which contained a permeable membrane coat having a weight ratio of water soluble, pH independent polymer:water insoluble, pH independent polymer of about 0.625:1, exhibited a release profile that was slightly slower than the release profile for the reference drug. Accordingly, the results of Example 6 indicate that cores containing a swellable, water soluble, pH independent polymer and a first water insoluble, pH independent polymer which have been coated with permeable membrane coatings containing a weight ratio of water soluble, pH independent polymer to a water insoluble, pH independent polymer between about 0.6:1 to about 1.2:1 are ideal for achieving an in vitro release profile similar to that of the reference drug.

### Example 7

Dosage forms were prepared to explore whether an in vitro release profile similar to the in vitro release profile for the 11 mg reference drug of Example 1 could be obtained with cores and permeable membrane coatings. Cores were prepared by blending 17.77 mg of tofacitinib citrate (equivalent to 11 mg tofacitinib), Polyox WSR N 80 (a swellable, water soluble, pH independent polymer) and Kollidon SR (a water insoluble, pH independent polymer) to form final blends. The final blends were combined with 4.0 mg of magnesium stearate and 4.0 mg of colloidal silicon dioxide, and then compressed to form cores. The cores were coated with one of two different coatings in the manner detailed above for Table 6. Each coating contained different amounts of HPMC E5 and Kollicoat SR 30 D. The coating formulations also contained triethyl citrate as a plasticizer, titanium dioxide as an opacifier, talc as a glidant. The content of the cores and coatings is detailed below in Table 11.

TABLE 11

| | | |
|---|---|---|
| Sample No. | 1 | 2 |
| Ingredients | mg/unit | mg/unit |

Core

| | | |
|---|---|---|
| Tofacitinib Citrate | 17.77 | 17.77 |
| Polyox N 80 | 147.77 | 152.77 |
| Kollidone SR | 30.46 | 25.46 |
| Colloidal silicon dioxide | 4 | 4 |
| Magnesium stearate | 4 | 4 |
| Sub-total (Core) | 204 | 204 |

Permeable Membrane Coating

| | | |
|---|---|---|
| Kollicoat SR 30 D | 3.06 | 4.16 |
| HPMC E 5 | 3.57 | 2.55 |
| Triethyl citrate | 0.51 | 0.51 |
| Titanium dioxide | 0.51 | 0.51 |
| Talc | 2.55 | 2.55 |
| Purified water | Q.S. | Q.S. |
| Total (Core + Coating) | 214.2 | 214.2 |

Both samples were subjected to the dissolution test detailed above for Example 1. The results obtained are set forth below in Table 12.

TABLE 12

| | Sample No. | |
|---|---|---|
| | (% drug dissolved) | |
| Time (hours) | 1 | 2 |
| 0 | 0 | 0 |
| 1 | 19.9 | 12.6 |
| 1.5 | 30.4 | 21.7 |
| 2 | 39.9 | 30.7 |
| 2.5 | 48.7 | 39.6 |
| 3 | 57.3 | 48.1 |
| 4 | 72.7 | 63.2 |
| 6 | 92.9 | 86.8 |
| 8 | 98.9 | 96.0 |

Similar to Sample 1 of Example 6, Sample 1 of Example 7 included a permeable membrane coat having a weight ratio of water soluble, pH independent polymer:water insoluble, pH independent polymer of about 1.167:1. Sample 1 of Example 7 exhibited a release profile that was slightly faster than the release profile for the 11 mg reference drug product. Sample 2 of Example 7, like Sample 2 of Example 6, included a permeable membrane coat having a weight ratio of water soluble, pH independent polymer:water insoluble, pH independent polymer of about 0.613:1. Sample 2 of Example 7 exhibited a release profile that was slightly slower than the release profile for the 11 mg reference drug. Thus, Example 7 demonstrates that cores containing a swellable, water soluble, pH independent polymer and a first water insoluble, pH independent polymer which have been coated with permeable membrane coatings containing a weight ratio of water soluble, pH independent polymer to a water insoluble, pH independent polymer between about 0.6:1 to about 1.2:1 are ideal for achieving in vitro release profiles similar to those of both the 11 mg and the 22 mg reference drugs.

### Example 8

Cores were prepared by blending 35.54 mg of tofacitinib citrate (equivalent to 22 mg tofacitinib), Polyox WSR N 80 LEO and Polyox WSR 205 LEO (swellable, water soluble, pH independent polymers), citric acid monohydrate (acidifier) and Kollidon SR (a water insoluble, pH independent polymer) to form final blends. In both cores, the amounts of Polyox WSR N 80 LEO and Polyox WSR 205 LEO were varied. The final blends were combined with 4.0 mg of magnesium stearate and 4.0 mg of colloidal silicon dioxide, and then compressed to form cores. Each core was coated with the same coating in the manner detailed above for Table 6. The coating contained HPMC E5, Kollicoat SR 30 D, triethyl citrate as a plasticizer, titanium dioxide as an opacifier, and talc as a glidant. A film coating, Opadry II Yellow, was applied to the coated cores after drying. The content of the cores and coatings is detailed below in Table 13.

TABLE 13

| | | |
|---|---|---|
| Sample No. | 1 | 2 |
| Ingredients | mg/unit | mg/unit |

Core

| | | |
|---|---|---|
| Tofacitinib Citrate | 35.54 | 35.54 |
| Polyox WSR N80 LEO | 97.47 | 51.98 |
| Polyox WSR 205 LEO | 32.49 | 77.98 |
| Citric acid monohydrate | 10.00 | 10.00 |
| Kollidone SR | 20.50 | 20.50 |
| Colloidal silicon dioxide | 4 | 4 |
| Magnesium stearate | 4 | 4 |
| Sub-total (Core) | 204 | 204 |

Permeable Membrane Coating

| | | |
|---|---|---|
| Kollicoat SR 30 D | 4.08 | 4.08 |
| HPMC E 5 | 2.55 | 2.55 |
| Triethyl citrate | 0.51 | 0.51 |
| Titanium dioxide | 0.51 | 0.51 |
| Talc | 2.55 | 2.55 |
| Purified water | Q.S. | Q.S. |
| Sub-total (Core + Coating) | 214.2 | 214.2 |
| Opadry II Yellow | 6.43 | 6.43 |
| Total | 220.63 | 220.63 |

Both samples were subjected to the dissolution test detailed above for Example 1, except that the paddles were rotated at 150 rpm. The results obtained are set forth below in Table 14.

TABLE 14

| | Sample No. | |
|---|---|---|
| | (% drug dissolved) | |
| Time (hours) | 1 | 2 |
| 0 | 0 | 0 |
| 1 | 10 | 9 |
| 1.5 | 20 | 18 |
| 2 | 34 | 29 |
| 2.5 | 48 | 42 |
| 3 | 62 | 55 |
| 4 | 82 | 77 |
| 6 | 97 | 101 |
| 8 | 102 | 102 |

The core formulations of Sample 1 and Sample 2 each contained a weight ratio of swellable, water soluble, pH independent polymer to water insoluble, pH independent polymer of 6.3:1. Sample 1, which contained 32.49 mg of a high viscosity and high molecular weight swellable, water soluble, pH independent polymer (Polyox WSR 205 LEO) and 97.47 mg of a low viscosity and low molecular weight water soluble, pH independent polymer (Polyox WSR N80 LEO), resulted in a release profile that was slightly faster compared to Sample 2 and similar to release profile for the reference drug product. In contrast, Sample 2, which contained a higher amount (77.98 mg) of Polyox WSR 205 LEO and a lower amount (51.98 mg) of Polyox WSR N80 LEO, exhibited a release profile that was slightly slower than the release profile for the reference drug. Accordingly, the results of Example 8 indicate that cores containing a mixture of a first swellable, water soluble, pH independent polymer having a low viscosity and a low molecular weight and a second swellable, water soluble, pH independent polymer having a high viscosity and high molecular weight in a weight ratio of about 1:0.25 to about 1:2.5 and a first water insoluble, pH independent polymer, and which have been coated with permeable membrane coatings containing a weight ratio of water soluble, pH independent polymer to a second water insoluble, pH independent polymer about 0.6:1 are also ideal for achieving an in vitro release profile similar to that of the reference drug.

### Example 9

A tablet core was prepared by blending 35.54 mg of tofacitinib citrate (equivalent to 22 mg tofacitinib), Polyox WSR N 80 LEO and Polyox WSR 205 LEO (swellable, water soluble, pH independent polymers), citric acid monohydrate (acidifier) and Kollidon SR (a water insoluble, pH independent polymer) to form a final blend. The final blend was combined with 4.0 mg of magnesium stearate and 4.0 mg of colloidal silicon dioxide, and then compressed to form a core. The core was coated with a similar permeable membrane coating in the manner detailed above for Table 6. The permeable membrane coating contained Methocel E5 LV (Hypromellose), Kollicoat SR 30 D, triethyl citrate as a plasticizer, titanium dioxide as an opacifier, and talc as a glidant. An enteric film coating was applied to the coated cores after drying. The enteric coating contained methacrylic acid copolymer NF (L30D55) as a pH dependent polymer, triethyl citrate as plasticizer, and talc as glidant. The enteric coating was prepared by dissolving triethyl citrate in purified water followed by the addition of methacrylic acid copolymer NF (L30D55) to form a polymer dispersion. Talc was screened through 60 mesh and added to the polymer dispersion to create the enteric coating dispersion. The enteric coating dispersion was applied to the coated cores by spray coating. The content of the cores and coatings is detailed below in Table 14.

TABLE 14

| | | |
|---|---|---|
| Sample No. | 1 | |
| Ingredients | mg/unit | % w/w |

Core

| | | |
|---|---|---|
| Tofacitinib Citrate | 35.54 | 16.11 |
| Polyox WSR N80 LEO | 97.47 | 44.18 |
| Polyox WSR 205 LEO | 32.49 | 14.73 |
| Citric acid monohydrate | 10.00 | 4.53 |
| Kollidone SR | 20.50 | 9.29 |
| Colloidal silicon dioxide | 4 | 1.81 |
| Magnesium stearate | 4 | 1.81 |
| Sub-total (Core) | 204 | |

Permeable Membrane Coating

| | | |
|---|---|---|
| Kollicoat SR 30 D | 4.08 | 1.85 |
| Methocel E5 (Premium) LV | 2.55 | 1.16 |
| Triethyl citrate | 0.51 | 0.23 |
| Titanium dioxide | 0.51 | 0.23 |
| Talc | 2.55 | 1.16 |
| Purified water | Q.S. | |
| Sub-total (Core + Coating) | 214.2 | |

Enteric Coating

| | | |
|---|---|---|
| L30D55 | 4.50 | 2.04 |
| Triethyl citrate | 0.64 | 0.29 |
| Talc | 1.28 | 0.58 |
| Purified water | Q.S. | |
| Total | 220.63 | 100.00 |

Many variations and modifications may be made to the above-described embodiments of the disclosure without departing substantially from the spirit and various principles of the disclosure. All such modifications and variations are intended to be included herein within the scope of this disclosure and protected by the following claims.

What is claimed is:

1. A sustained release pharmaceutical dosage form comprising:
a core comprising:
tofacitinib or a pharmaceutically acceptable salt thereof;
a swellable, water soluble, pH independent polymer, wherein the swellable, water soluble, pH independent polymer has a molecular weight between about 150,000 to about 750,000; and
a first water insoluble, pH independent polymer;
and a permeable membrane coat surrounding the core, the coat comprising:
a water soluble, pH independent polymer; and
a second water insoluble, pH independent polymer;
wherein the weight ratio of the water soluble, pH independent polymer to the second water insoluble, pH independent polymer in the permeable membrane coat is from about 0.4:1 to about 1.5:1.

2. The dosage form of claim 1, wherein the swellable, water soluble, pH independent polymer of the core is selected from the group consisting of polyethylene oxide; glyceryl fatty acid esters; hydrogenated castor oil; hydroxyethyl cellulose; hydroxypropyl cellulose; hydroxypropyl methylcellulose; ethylhydroxy ethylcellulose; methylethyl cellulose; carboxymethyl cellulose; carboxymethyl ethylcellulose; pullulan; polyvinyl pyrrolidone; polyvinyl alcohol; polyvinyl acetate and mixtures thereof.

3. The dosage form of claim 1, wherein the water soluble, pH independent polymer of the permeable membrane coat is selected from the group consisting of polyethylene oxide; glyceryl fatty acid esters; hydrogenated castor oil; hydroxyethyl cellulose; hydroxypropyl cellulose; hydroxypropyl methylcellulose; ethylhydroxy ethylcellulose; methylethyl cellulose; carboxymethyl cellulose; carboxymethyl ethylcellulose; pullulan; polyvinyl pyrrolidone; polyvinyl alcohol; polyvinyl acetate; ethyl acrylate; methyl methacrylate; and mixtures thereof.

4. The dosage form of claim 1, wherein the first water insoluble, pH independent polymer is selected from the group consisting of copolymers of methacrylic acid or methacrylic acid esters; polyvinyl chloride; polyethylene; cellulose; cellulose derivatives; polyvinyl alcohol phthalate; polyvinyl acetate phthalate; polyvinylbutyl phthalate; polyvinyl acetate; polyvinyl acetate copolymers; crosslinked polyvinylpyrrolidone; carnauba wax; microcrystalline wax; triglycerides; and mixtures thereof.

5. The dosage form of claim 1, wherein the second water insoluble, pH independent polymer is selected from the group consisting of copolymers of methacrylic acid or methacrylic acid esters; polyvinyl chloride; polyethylene; cellulose; cellulose derivatives; polyvinyl alcohol phthalate; polyvinyl acetate phthalate; polyvinylbutyl phthalate; polyvinyl acetate; polyvinyl acetate copolymers; crosslinked polyvinylpyrrolidone; carnauba wax; microcrystalline wax; triglycerides; and mixtures thereof.

6. The dosage form of claim 1, wherein the first water insoluble, pH independent polymer and the second water insoluble, pH independent polymer are different from one another.

7. The dosage form of claim 1, wherein the swellable, water soluble, pH independent polymer is present in the core in an amount of about 60% to about 75% by weight, based on the total weight of the core.

8. The dosage form of claim 1, wherein the first water insoluble, pH independent polymer is present in the core in an amount of from about 5% to about 20% by weight, based on the total weight of the core.

9. The dosage form of claim 1, wherein the weight ratio of the swellable, water soluble, pH independent polymer to the first water insoluble, pH independent polymer in the core is from about 3:1 to about 10:1.

10. The dosage form of claim 1, wherein the permeable membrane coat comprises about 3% to about 10% weight percent, based on the total weight of the dosage form and the water soluble, pH independent polymer is present in the permeable membrane coat in an amount of from about 20% to about 40% weight percent, based on the total weight of the permeable membrane coat.

11. The dosage form of claim 10, wherein the weight ratio of the core in an uncoated state to the permeable membrane coat is about 15:1 to about 25:1, and the ratio of the swellable, water soluble, pH independent polymer to the first water insoluble, pH independent polymer of the core is about 3:1 to about 10:1.

12. The dosage form of claim 1, wherein the sustained release dosage form comprises from about 11 mg to about 22 mg of tofacitinib or an equivalent amount of a citrate salt thereof, and
the sustained release dosage form releases about 20% to about 40% of tofacitinib or an equivalent amount of a citrate salt thereof when the sustained release dosage form is subjected to a dissolution test at 50 rpm in 0.05M pH 6.8 potassium phosphate buffer at 37° C. for two hours.

13. The dosage form of claim 12, wherein the sustained release dosage form releases about 85% to about 100% of tofacitinib or an equivalent amount of a citrate salt thereof when the sustained release dosage form subjected to the dissolution test after six hours.

14. The dosage form of claim 1, wherein the sustained release dosage form delivers tofacitinib or a pharmaceutically acceptable salt thereof to a subject by diffusion through the permeable membrane coat, wherein the permeable membrane coat covers the entire surface of the core.

15. The dosage form of claim 14, wherein the sustained release dosage form provides a zero-order release profile when administered to a subject.

16. The dosage form of claim 1, wherein the sustained release dosage form is free of an osmagen, a diluent, or a combination thereof.

17. The dosage form of claim 1, wherein the core further comprises a second swellable, water soluble, pH independent polymer, wherein the second swellable, water soluble, pH independent polymer has a molecular weight between about 150,000 to about 750,000.

18. The dosage form of claim 17, wherein the swellable, water soluble, pH independent polymer has a molecular weight between about 150,000 to about 250,000 and the second swellable, water soluble, pH independent polymer has a molecular weight between about 500,000 to about 750,000.

19. The dosage form of claim 17, wherein the swellable, water soluble, pH independent polymer has a viscosity between about 25 cP and about 500 cP at room temperature, and the second swellable, water soluble, pH independent polymer has a viscosity between about 4,000 cP and about 10,000 cP at room temperature.

20. The dosage form of claim 1, further comprising an enteric coating surrounding the permeable membrane coat on the core, the enteric coating comprises about 1% to about 5% weight percent, based on the total weight of the dosage form.
